(19) United States
(12) Patent Application Publication
(10) Pub. No.: US 2024/0180448 A1
(43) Pub. Date: Jun. 6, 2024

(54) METHOD OF MONITORING BLOOD SUGAR AND ELECTRONIC DEVICE SUPPORTING SAME

(71) Applicant: Samsung Electronics Co., Ltd., Suwon-si (KR)

(72) Inventor: Hyejung SEO, Suwon-si (KR)

(21) Appl. No.: 18/397,321

(22) Filed: Dec. 27, 2023

Related U.S. Application Data
(63) Continuation of application No. PCT/KR2023/019814, filed on Dec. 4, 2023.

(30) Foreign Application Priority Data
Dec. 5, 2022 (KR) 10-2022-0167726
Jan. 19, 2023 (KR) 10-2023-0008087

(51) Int. Cl. A61B 5/145; A61B 5/00

(52) U.S. Cl. CPC A61B 5/14532; A61B 5/681; A61B 5/7282

(57) ABSTRACT

A wearable electronic device is provided. The wearable electronic device includes a first sensor, a second sensor, a memory, and a processor, wherein the processor may be configured to measure a blood sugar level in a first cycle using the first sensor so as to store a first blood sugar level measured at a first time point, detect a specified event, based on measured values received from the second sensor from a second time point after the first time point to a third time point, if the specified event is detected, measure a blood sugar level of the user in a second cycle different from the first cycle so as to store at least one second blood sugar level measured from a fourth time point to a fifth time point according to the second cycle, and calculate at least one feature value related to blood sugar of the user.

### CROSS-REFERENCE TO RELATED APPLICATION(S)

This application is a continuation application, claiming priority under § 365(c), of an International application No. PCT/KR2023/019814, filed on Dec. 4, 2023, which is based on and claims the benefit of a Korean patent application number 10-2022-0167726, filed on Dec. 5, 2022, in the Korean Intellectual Property Office, and of a Korean patent application number 10-2023-0008087, filed on Jan. 19, 2023, in the Korean Intellectual Property Office, the disclosure of each of which is incorporated by reference herein in its entirety.

### TECHNICAL FIELD

The disclosure relates to a method of monitoring blood sugar and an electronic device supporting the same.

### BACKGROUND ART

In recent years, interest in health has increased in line with great enhancement of the living environment and improvement in the quality of life. Accordingly, development of medical user equipment capable of easily checking health conditions is actively progressing.

Meanwhile, the concentration of glucose (hereinafter, referred to as blood sugar) in a human body fluid (e.g., blood or urine) is an important criterion for indicating a person's health condition. For example, excessively high or low blood sugar levels can be fatal to personal health. Therefore, continuous monitoring of personal blood sugar may be an important factor to check the personal health conditions.

Methods of measuring blood sugar include an invasive method in which blood sugar is directly collected and measured, and a non-invasive method in which blood sugar is measured without collection. A device for measuring blood sugar in an invasive method may include a blood collection-type device in which blood is collected and measured directly from a fingertip by a user and a continuous blood sugar monitoring device in which a microneedle is inserted into the user's abdomen or upper arm to measure blood sugar. A device for measuring blood sugar in a non-invasive method may transmit an electromagnetic wave (e.g., light, radio wave, or sound) signal into the human body from the outside and measure the amount of change in the signal magnitude or phase due to the reaction of blood sugar.

The above information is presented as background information only to assist with an understanding of the disclosure. No determination has been made, and no assertion is made, as to whether any of the above might be applicable as prior art with regard to the disclosure.

### DISCLOSURE

### Technical Problem

A blood collection-type device among the invasive blood sugar measurement devices has difficulty in monitoring the amount of change in blood sugar and the direction of change thereof because the user suffers from pain during blood collection, cost is incurred for consumables such as blood sugar strips or blood collection needles, and the blood sugar level is recorded only at the time of measurement. In addition, although a continuous blood sugar monitoring device among the invasive blood sugar measurement devices enables periodical measurement at regular time intervals, secondary diseases such as skin irritation and inflammation may occur in the process of inserting a microneedle into the skin and expensive sensors thereof are required to be replaced for a specific period, causing an economic burden.

Aspects of the disclosure are to address at least the above-mentioned problems and/or disadvantages and to provide at least the advantages described below. Accordingly, an aspect of the disclosure is to provide a method of monitoring blood sugar by continuously (or repetitively) measuring blood sugar in a non-invasive manner and an electronic device supporting the same.

Another aspect of the disclosure is to provide a method of monitoring blood sugar by switching a blood sugar measurement mode, based on a user's context, and an electronic device supporting the same.

Another aspect of the disclosure is to provide a method of monitoring blood sugar by utilizing a blood sugar level measured immediately before detection of a specified event as an initial value in the blood sugar change when the specified event is detected, and an electronic device supporting the method.

Another aspect of the disclosure is to provide a method of monitoring blood sugar by adjusting a blood sugar measurement cycle, based on a result of comparing a feature value related to blood sugar with a threshold, and an electronic device supporting the same.

Additional aspects will be set forth in part in the description which follows and, in part, will be apparent from the description, or may be learned by practice of the presented embodiments.

### Technical Solution

In accordance with an aspect of the disclosure, a wearable electronic device is provided. The wearable electronic device includes a first sensor, a second sensor, a memory, and a processor operatively connected to the first sensor, the second sensor, and the memory. The processor is configured to measure a blood sugar level of a user in a first cycle using the first sensor so as to store a first blood sugar level measured at a first time point according to the first cycle in the memory, detect a specified event, based on measured values received from the second sensor from a second time point after the first time point to a third time point, measure, if the specified event is detected, a blood sugar level of the user in a second cycle different from the first cycle using the first sensor so as to store at least one second blood sugar level measured from a fourth time point to a fifth time point according to the second cycle in the memory, and calculate at least one feature value related to blood sugar of the user, based on the first blood sugar level and the at least one second blood sugar level.

In accordance with another aspect of the disclosure, a blood sugar monitoring method is provided. The blood sugar monitoring method includes measuring a blood sugar level of a user in a first cycle using a first sensor so as to store a first blood sugar level measured at a first time point according to the first cycle in a memory, detecting a specified event, based on measured values received from a second sensor from a second time point after the first time point to a third time point, if the specified event is detected, measuring a blood sugar level of the user in a second cycle different from the first cycle using the first sensor so as to store at least one second blood sugar level measured from a fourth time point to a fifth time point according to the second cycle in the memory, and calculating at least one feature value related to blood sugar of the user, based on the first blood sugar level and the at least one second blood sugar level.

### Advantageous Effects

According to various embodiments of the disclosure, blood sugar may be continuously (or repeatedly) measured in a non-invasive manner, enabling consistent monitoring of blood sugar without causing pain to the user and without incurring cost for consumables.

In addition, according to various embodiments of the disclosure, a blood sugar measurement mode may be switched based on the user's context, thereby more accurately monitoring blood sugar depending on the user's context.

In addition, according to various embodiments of the disclosure, when a specified event is detected, a blood sugar level measured immediately before detecting the specified event may be used as an initial value in the blood sugar change, thereby more accurately determining the user's health conditions according to the blood sugar change.

In addition, according to various embodiments of the disclosure, a blood sugar measurement cycle may be adjusted based on a result of comparing a feature value related to blood sugar with a threshold, thereby more accurately judging the user's health conditions depending on the user's context.

In addition, various effects identified directly or indirectly through this document may be provided.

Other aspects, advantages, and salient features of the disclosure will become apparent to those skilled in the art from the following detailed description, which, taken in conjunction with the annexed drawings, discloses various embodiments of the disclosure.

### DESCRIPTION OF DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment according to an embodiment of the disclosure;

FIG. 2 is a diagram illustrating an electronic device for monitoring blood sugar according to an embodiment of the disclosure;

FIG. 3 is a diagram illustrating a blood sugar curve according to an embodiment of the disclosure;

FIG. 4 is a diagram illustrating a relationship between detection of a specified event and a change in blood sugar according to an embodiment of the disclosure;

FIG. 5 is a diagram illustrating a relationship between a detection cycle of a specified event and a blood sugar measurement cycle according to an embodiment of the disclosure;

FIG. 6 is a diagram illustrating a method of adjusting a blood sugar measurement cycle according to detection of a specified event according to an embodiment of the disclosure;

FIG. 7 is a flowchart illustrating a method of monitoring blood sugar, based on a user's context, according to an embodiment of the disclosure;

FIG. 8 is a flowchart illustrating a method of monitoring blood sugar when a specified event is detected according to an embodiment of the disclosure; and

FIG. 9 is a flowchart illustrating a method of monitoring blood sugar when a specified event is detected according to an embodiment of the disclosure.

Throughout the drawings, like reference numerals will be understood to refer to like parts, components, and structures.

### MODE FOR INVENTION

The following description with reference to the accompanying drawings is provided to assist in a comprehensive understanding of various embodiments of the disclosure as defined by the claims and their equivalents. It includes various specific details to assist in that understanding, but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but are merely used by the inventor to enable a clear and consistent understanding of the disclosure. Accordingly, it should be apparent to those skilled in the art that the following description of various embodiments of the disclosure is provided for illustration purposes only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

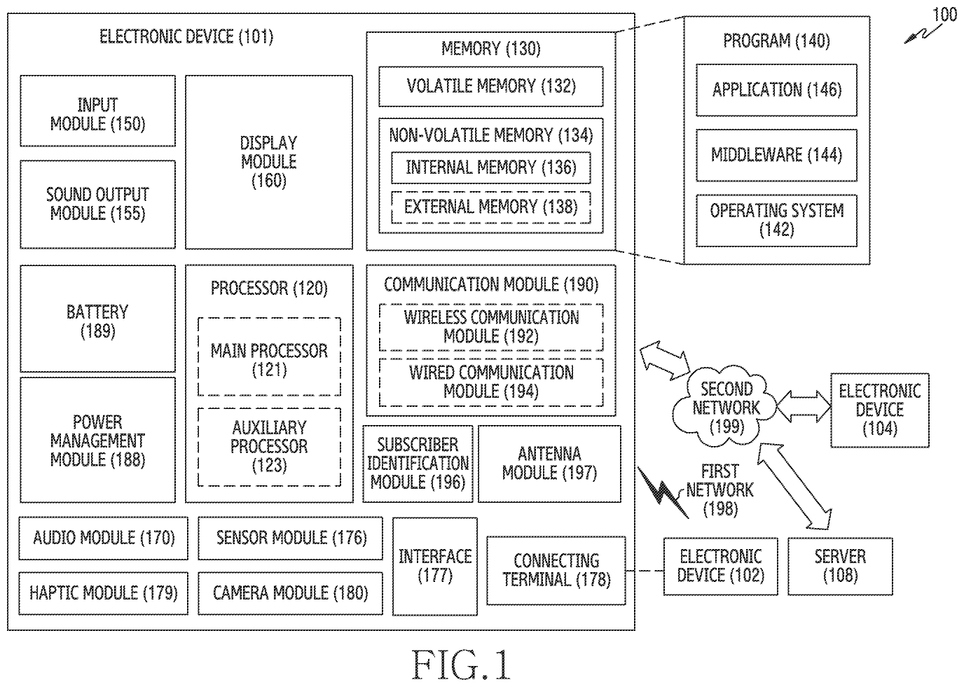

FIG. 1 is a block diagram illustrating an electronic device 101 in a network environment 100 according to an embodiment of the disclosure.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5th generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4th generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 gigabits per second (Gbps) or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IOT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

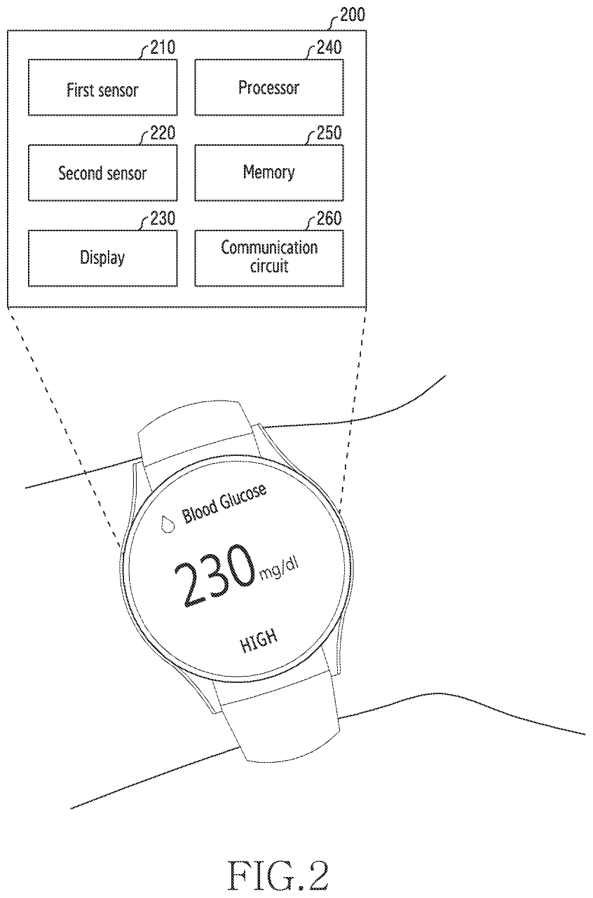

FIG. 2 is a diagram illustrating an electronic device for monitoring blood sugar according to an embodiment of the disclosure.

Referring to FIG. 2, an electronic device 200 (e.g., the electronic device 101 in FIG. 1) for monitoring blood sugar may include a wearable device. The wearable device is a device that may be worn by a user and may include at least one of an accessory-type device (e.g., a watch, a ring, a bracelet, an anklet, a necklace, glasses, or contact lenses), a head-mounted device (HMD), a fabric or clothing-integrated device (e.g., an electronic garment), a body-attachable device (e.g., a skin pad), or a bio-implantable device (e.g., an implantable circuit). However, the type of wearable device is not limited thereto. The electronic device 200 for monitoring blood sugar is not limited to the wearable devices above. The electronic device 200 for monitoring blood sugar may be any device capable of measuring blood sugar in a target subject (e.g., a user's skin tissue) in a non-invasive manner.

The electronic device 200 for monitoring blood sugar may include a first sensor 210 (e.g., the sensor module 176 in FIG. 1), a second sensor 220 (e.g., the sensor module 176 in FIG. 1), and a display 230 (e.g., the display module 160 in FIG. 1), a processor 240 (e.g., the processor 120 in FIG. 1), a memory 250 (e.g., the memory 130 in FIG. 1), and a communication circuit 260 (e.g., the communication module 190 in FIG. 1). However, the configuration of the electronic device 200 is not limited thereto. According to various embodiments, the electronic device 200 may omit at least one of the above-described elements and further include at least one other element not shown in FIG. 2.

The first sensor 210 may measure blood sugar in the target subject in a non-invasive manner. The first sensor 210 may include an optical sensor. The optical sensor may emit light (e.g., visible light, near-infrared light, or mid-infrared light) to the test subject, receive at least some of reflected light, and analyze the received light to calculate a blood sugar level. The first sensor 210 may include two or more electrodes. The first sensor 210 may apply current through the electrodes in the state in which the electrodes are in contact with the test subject and analyze a change in the returned current, thereby calculating a blood sugar level. The first sensor 210 may include a photoplethysmography (PPG) sensor. The PPG sensor is a type of the optical sensor and may radiate light in a specified frequency band to a test subject, receive at least some of a reflected light thereof, and analyze the received light to calculate a change in blood volume in a blood vessel, oxygen saturation, and/or a blood sugar level. The first sensor 210 may include an acoustic sensor, an ultrasonic sensor, or a heat flux sensor. The first sensor 210 may include one or more of the above-described sensors. For example, the first sensor 210 may include at least one of the optical sensor, the electrodes, the PPG sensor, the acoustic sensor, the ultrasonic sensor, or the heat flux sensor. The first sensor 210 may include a plurality of sensors from among the above-described sensors. In this case, at least one (e.g., an optical sensor or an ultrasonic sensor using mid-infrared rays) of the plurality of sensors may activate blood sugar molecules of the user. In addition, at least one other sensor (e.g., an optical sensor using infrared rays) among the plurality of sensors may emit light and measure a change in the refractive index of light due to thermal energy generated from the activated blood sugar molecules to calculate the blood sugar level.

The second sensor 220 may be used to determine the user's context. The user's context may include at least one of a user's biometric information, a user's situation, or a user's external environment. The second sensor 220 may include at least one of a PPG sensor, a temperature/humidity sensor, an altitude sensor, an electrode sensor, a motion sensor, an acceleration sensor, a proximity sensor, a gyro sensor, an iris sensor, an illuminance sensor, a pressure sensor, a time-of-flight (TOF) sensor, or a microphone. The second sensor 220 may be included in an external electronic device.

The display 230 may display a variety of content (e.g., text, images, videos, icons, or symbols) to the user. The display may include a touch screen and receive at least one of a touch, gesture, proximity, or hovering input using an electronic pen or a user's body part. The display 230 may display at least one of the user's blood sugar level measured through the first sensor 210, information about the user's context determined based on the measured value of the second sensor 220, or information about the user's health conditions determined based on the user's blood sugar level according to the user's context.

The processor 240 may control at least one element of the electronic device 200 and perform processing a variety of data or calculations. The processor 240 may perform functions related to blood sugar monitoring by executing instructions stored in the memory 250.

The processor 240 may measure the user's blood sugar level using the first sensor 210. The processor 240 may measure the user's blood sugar level in a first cycle using the first sensor 210. In addition, the processor 240 may store, in the memory 250, a first blood sugar level measured at a first time point according to the first cycle. The first time point may be, for example, a time point in a user's fasting state. The first cycle may be, for example, a 15-minute cycle.

The processor 240 may determine the user's context, based on a measured value of the second sensor 220. The processor 240 may detect a specified event, based on measured values received from the second sensor 220 from a second time point after the first time point to a third time point. The specified event may correspond to an event indicating that the user starts eating food (hereinafter referred to as a food intake start event). The processor 240 may detect an event subsequent to the specified event, based on measured values received from the second sensor 220. The subsequent event may correspond to an event indicating that the user has finished eating food (hereinafter referred to as a food intake end event).

The processor 240 may use a machine learning technique to detect an event related to food intake (e.g., the food intake start event and/or the food intake end event). For example, the processor 240 may divide an inertia signal generated by a user's motion and use arithmetic statistics thereof as feature points in order to learn a model for detecting the user's food intake action. The processor 240 may classify the user's actions in consideration of weights for specific motions (e.g., feature points) according to the principle of the learning model. In addition, the processor 240 may add data for learning and repeatedly perform learning in order to improve the classified model. The processor 240 may select structural feature points of a waveform of a measurable motion sensor from detailed actions in which an event related to food intake may occur and classify the food intake actions of the user using relative positions and states between the respective feature points. The food intake action may include processes of picking up food, moving the arm to put the food in the mouth, and lowering back the arm by the user. The processor 240 may utilize data such as acceleration signal data measured during the user's action of eating food, change data in the direction of gravity generated while rotating the wrist, or a change in feature points of a gyro signal. The processor 240 may use at least one of the average value, standard deviation, maximum value, minimum value, mode value, or median value for each axis of the acceleration sensor and/or the gyro sensor at minimum unit time (e.g., 1 minute) intervals in order to use statistical features. In addition, the processor 240 may utilize various machine learning techniques such as temporal convolutional network (TCN), decision tree (DT), and k-nearest neighbors (KNN).

The processor 240 may use a microphone sensor (e.g., the input module 150 in FIG. 1) in order to detect an event related to food intake (e.g., the food intake start event and/or the food intake end event). For example, the processor 240 may obtain sound data generated when the user eats food using the microphone sensor and detect an event related to food intake using the sound data. As another example, the processor 240 may detect an event related to food intake using sound data, which is generated when the user eats food, obtained using the microphone sensor and data on the movement of the user's temporomandibular joint while chewing the food measured by an acceleration sensor.

If the specified event (e.g., the food intake start event) is detected, the processor 240 may measure the user's blood sugar level in a second cycle, which is different from the first cycle, using the first sensor 210. For example, the processor 240 may change (or adjust) the measurement cycle (e.g., the blood sugar measurement cycle) of the first sensor 210 from the first cycle to the second cycle. In addition, the processor 240 may store, in the memory 250, at least one second blood sugar level measured from a fourth time point to a fifth time point according to the second cycle. The period from the fourth time point to the fifth time point may be, for example, one of periods corresponding to the state in which the user is eating food and the state in which a predetermined time elapses after the user eats food. The second cycle may be shorter than the first cycle. The second cycle may be, for example, a 5-minute cycle.

The processor 240 may calculate at least one feature value related to the user's blood sugar, based on the first blood sugar level and the at least one second blood sugar level. The first blood sugar level is a blood sugar level measured in the first cycle at the first time point and may correspond to, for example, the fasting blood sugar level measured on an empty stomach. The at least one second blood sugar level is a blood sugar level measured in the second cycle from the fourth time point to the fifth time point and may correspond to, for example, a blood sugar level measured during food intake or a postprandial blood sugar level. The fourth time point at which the at least one second blood sugar level starts to be measured may be the same as the third time point or later than that in consideration of the time taken to detect the specified event. The specified event may be detected at a time point somewhat later than the time point at which the user actually starts to eat food. For example, the blood sugar level measured at the fourth time point may be the blood sugar level measured after the user has consumed food to some extent, instead of the blood sugar level at the time point when the user actually starts to eat food. Accordingly, in order to more accurately monitor a change in blood sugar according to the occurrence (or detection) of the specified event, the processor 240 may configure an initial value of the change in blood sugar of the user according to the specified event as the first blood sugar level.

The at least one feature value related to the user's blood sugar may include at least one of a current blood sugar level, a fasting blood sugar level, a rate of change in blood sugar, a direction of change in blood sugar, the blood sugar response area (the area under the curve (AUC)), a slope of a blood sugar rise curve, a maximum blood sugar level (or blood sugar peak value), a change in blood sugar before and after eating food, the time required to return to the fasting blood sugar after eating food, the number of blood sugar peaks, or occurrence intervals of food intake events.

The processor 240 may configure a threshold for the at least one feature value. The processor 240 may configure a threshold of the current blood sugar level as 200 mg/dl for a user input value or a user not diagnosed with diabetes. The processor 240 may configure a threshold for the fasting blood sugar level as 100 mg/dl for a user input value or a user not diagnosed with diabetes. The processor 240 may configure a threshold for the blood sugar change rate as 1 mg/dl/min. The processor 240 may configure a threshold for the maximum blood sugar level as 200 mg/dl for a user input value or a user not diagnosed with diabetes. The processor 240 may configure a threshold for the amount of change in blood sugar before and after eating food as 100 mg/dl. The processor 240 may configure a threshold for the time required to return to fasting blood sugar after eating food as 2 hours after eating. The processor 240 may configure a threshold for the number of blood sugar peaks as the number of occurrences (or detections) of the specified event.

The processor 240 may compare the at least one feature value with the threshold and, based on a result of the comparison, change the measurement cycle of the first sensor 210 to a third cycle, which is different from the second cycle. If the at least one feature value exceeds the threshold, the processor 240 may change the measurement cycle of the first sensor 210 to a third cycle different from the second cycle. If the number of times the at least one feature value exceeds the threshold exceeds a specified number of times during a specified period, the processor 240 may change the measurement cycle of the first sensor 210 to a third cycle different from the second cycle. The third cycle may be shorter than the second cycle. The third cycle may be a 1-minute cycle.

If a third blood sugar level of the user measured using the first sensor 210 is equal to or less than a threshold when a specified time elapses after the specified event is detected, the processor 240 may change the measurement cycle of the first sensor 210 from the second cycle to the first cycle. For example, if a third blood sugar level of the user measured using the first sensor 210 is recovered to 140 mg/dl or less when a specified time (e.g., 2 hours) elapses after eating, the processor 240 may change the measurement cycle of the first sensor 210 back to the first cycle.

The processor 240 may compare the at least one feature value with the threshold and, based on a result of comparing the at least one feature value with the threshold, determine whether or not to provide a notification related to the user's blood sugar. If the at least one feature value exceeds the threshold, the processor 240 may determine whether or not to provide a notification related to the user's blood sugar. If the number of times the at least one feature value exceeds the threshold exceeds a specified number of times during a specified period, the processor 240 may determine whether or not to provide a notification related to the user's blood sugar.

The notification may include, information about the user's health condition and information related to a diet or exercise guide determined based on the at least one feature value.

The memory 250 may store a variety of data used by at least one element of the electronic device 200. The memory 250 may store instructions and data related to blood sugar monitoring. In this case, the instructions may be executed by the processor 240.

The communication circuit 260 may support communication between the electronic device 200 and an external electronic device. For example, the communication circuit 260 may establish wired or wireless communication with an external electronic device according to prescribed communication protocol and transmit/receive signals or data.

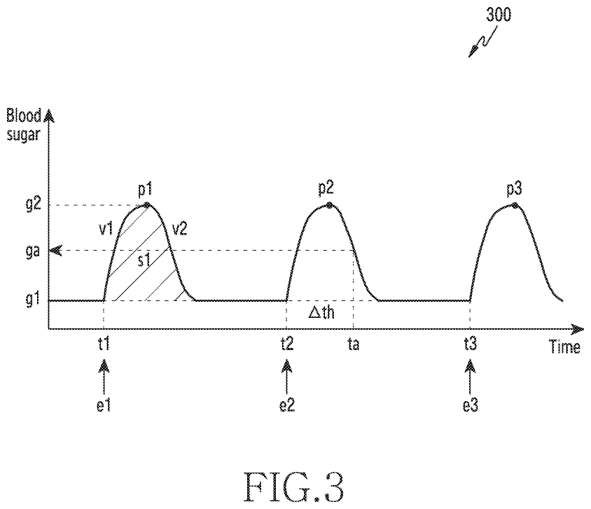

FIG. 3 is a diagram illustrating a blood sugar curve according to an embodiment of the disclosure.

Referring to FIG. 3, a blood sugar curve graph 300 may represent changes in blood sugar depending on food intake. When a certain amount of time (e.g., 8 hours) elapses from the previous food intake time, the user may be in a fasting state, which may satisfy the condition for measuring the fasting blood sugar level. For example, in the blood sugar curve graph 300, a blood sugar level g1 measured prior to a first time t1 may correspond to the fasting blood sugar level. A processor (e.g., the processor 240 in FIG. 2) of an electronic device (e.g., the electronic device 200 in FIG. 2) may monitor a trend of the fasting blood sugar level measured in the fasting state. Since the greater the degree of change in fasting blood sugar, the lower the pancreatic function and the greater the risk of diabetes, the processor may continuously monitor the fasting blood sugar level. In addition, if the fasting blood sugar greatly increases and changes, the processor may provide a notification to the user and provide information for managing blood sugar (e.g., information related to a diet or exercise guide).

If it is determined that the user is in the fasting state, the processor may switch to a measurement mode (e.g., a fasting blood sugar test mode) for more precisely measuring the fasting blood sugar level. According to an embodiment, in the fasting blood sugar test mode, the processor may quickly configure a measurement cycle of a first sensor (e.g., the first sensor 210 in FIG. 2) of the electronic device. In the fasting blood sugar test mode, in order to measure the fasting blood sugar level more precisely, the processor may identify a measured value having the best signal quality when preprocessing measured values of the first sensor and store (or record) the identified measured value as the fasting blood sugar level together with context information indicating the fasting state. The processor may use a threshold (or threshold range) (e.g., a signal quality index) configure according to the type of the first sensor in the process of identifying a measured value having the best signal quality from among the measured values of the first sensor. For example, the processor may identify the largest value that does not exceed the threshold, among the measured values of the first sensor, as the measured value having the best signal quality.

If a food intake event occurs (or is detected), in general, the blood sugar level may drop below 140 mg/dl within a certain period of time (e.g., 2 hours) after the food intake and may not exceed 200 mg/dl at the maximum, and the maximum blood sugar level may be measured between 30 minutes and 1 hour after the food intake. The blood sugar level may rise after food intake and then slowly fall back to the normal fasting blood sugar level. For example, if food intake start events e1, e2, and e3 are detected at a first time t1, a second time t2, or a third time t3 in the blood sugar curve graph 300, the blood sugar level may rise at a first rate v1 (or blood sugar increase rate) to be measured up to the maximum blood sugar level g2 between 30 minutes and 1 hour, and may relatively slowly decrease to the normal fasting blood sugar level at a second rate v2 (or blood sugar decrease rate). At this time, the points at which the blood sugar level becomes the maximum may be referred to as blood sugar peaks p1, p2, and p3, and the number of blood sugar peaks may be the same as the number of occurrences (or detections) of food intake events. Here, the blood sugar level ga measured at a time point ta after a certain period of time (Δth=ta−t2) (e.g., 2 hours) elapses from the time point t2 of food intake may correspond to the postprandial blood sugar level. If the postprandial blood sugar level is not 140 mg/dl or less, the processor may configure the measurement cycle of the first sensor to be shorter. In addition, when the postprandial blood sugar level is not 140 mg/dl or less, the processor may provide the user with a notification and information for blood sugar management (e.g., information related to a diet or exercise guide). In addition, the processor may restore the measurement cycle of the first sensor if the postprandial blood sugar level is 140 mg/dl or less.

The processor may detect a food intake start event, based on at least one of an increase in blood sugar level to a predetermined level or more (e.g., 1.5 mg/dl/min or more), a measurement time, a fasting blood sugar level, and the blood sugar response area s1, a heart rate, or an event related to previous food intake, during continuous (or repetitive) blood sugar monitoring. In addition, if a food intake start event is detected, the processor may switch to a measurement mode (e.g., glucose tolerance test mode) for precisely measuring the blood sugar level. For example, the processor may configure the measurement cycle of the first sensor of the electronic device to be shorter.

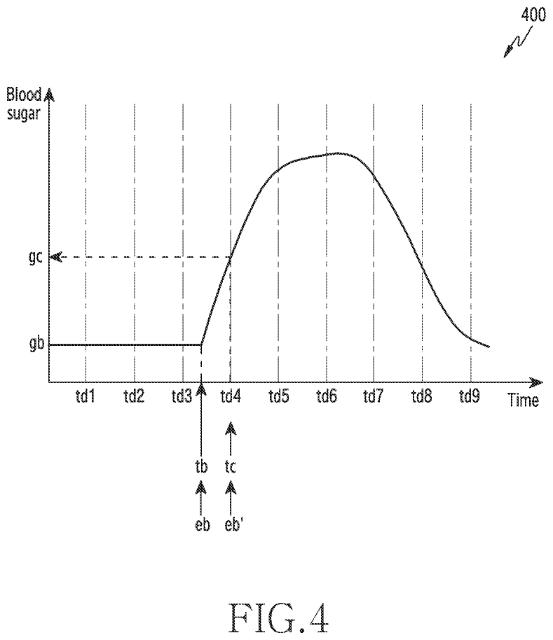

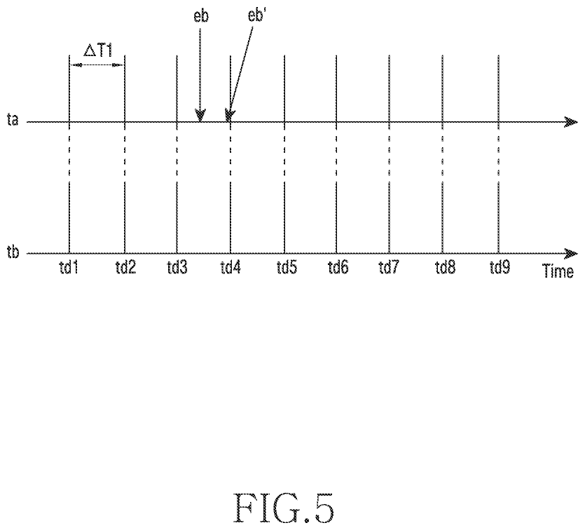

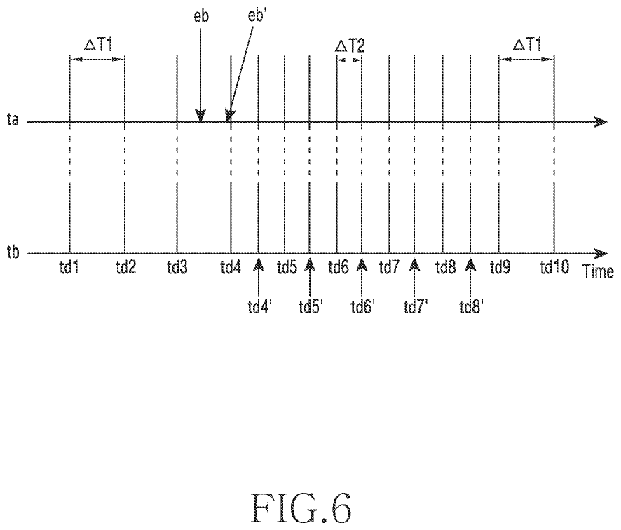

FIG. 4 is a diagram illustrating a relationship between detection of a specified event and a change in blood sugar according to an embodiment of the disclosure, FIG. 5 is a diagram illustrating a relationship between a detection cycle of a specified event and a blood sugar measurement cycle according to an embodiment of the disclosure, and FIG. 6 is a diagram illustrating a method of adjusting a blood sugar measurement cycle according to detection of a specified event according to an embodiment of the disclosure.

Referring to FIGS. 4 to 6, the blood sugar curve graph 400 may represent changes in blood sugar depending on food intake. A processor (e.g., the processor 240 in FIG. 2) of an electronic device (e.g., the electronic device 200 in FIG. 2) may measure a blood sugar level using a first sensor (e.g., the first sensor 210 in FIG. 2). For example, the processor may measure at least one first blood sugar level at a first time point td1, a second time point td2, and a third time point td3 according to a first cycle ΔT1 using the first sensor. The at least one first blood sugar level may correspond to a fasting blood sugar level.

The processor may detect a specified event (e.g., a food intake start event), based on a measured value of a second sensor (e.g., the second sensor 220 in FIG. 2). For example, the processor may detect the specified event eb′, based on measured values received from the second sensor from a 3-1st time point tb to a 3-2nd time point tc after the third time point td3.

If the specified event eb′ is detected, the processor may measure the user's blood sugar level using the first sensor. For example, as shown in FIG. 5, the processor may measure at least one second blood sugar level at a fourth time point td4, a fifth time point td5, a sixth time point td6, a seventh time point td7, an eighth time point td8, and a ninth time point td9 according to the first cycle ΔT1 using the first sensor. The at least one second blood sugar level may correspond to a blood sugar level measured during food intake or a postprandial blood sugar level. Considering the time (tc-tb) required to detect the specified event eb′, the fourth time point td4 at which the at least one second blood sugar level starts to be measured may be the same as the 3-2nd time point tc or may be later than the 3-2nd time point tc. The time point tc at which the specified event eb′ is detected may be somewhat later than the time point tb at which the user actually starts to eat food (the actual event eb occurs). For example, the blood sugar level gc measured at the fourth time point td4 may be a blood sugar level measured after the user has consumed some food and may not be the blood sugar level at the time point tb at which the user actually starts to eat food. Accordingly, in order to more accurately monitor a change in blood sugar according to the occurrence (or detection) of the specified event eb′, the processor may configure an initial value of the change in the user's blood sugar according to the specified event eb′ as a last measured first blood sugar level (e.g., the blood sugar level gb measured at the third time point td3) from among the at least one first blood sugar level.

If the specified event eb′ is detected, the processor may measure a user's blood sugar level in a second cycle ΔT2, which is different from the first cycle ΔT1, using the first sensor. For example, as shown in FIG. 6, the processor, using the first sensor, at least one second blood sugar level at a 4th time point td4, a 4-1st time point td4′, a 5th time point td5, a 5-1st time point td5, a 6th time point td6, a 6-1st time point td6′, a 7th time point td7, a 7-1st time point td7′, an 8th time point td8, an 8-1st time point td8′, and a 9th time point td9 according to the second cycle ΔT2. The processor may change (or adjust) a measurement cycle Tb (e.g., a blood sugar measurement cycle) of the first sensor from the first cycle ΔT1 to the second cycle ΔT2. The processor may change (or adjust) a measurement cycle Ta (e.g., an event detection cycle) of the second sensor from the first cycle T1 to the second cycle ΔT2.

If the user's blood sugar level (e.g., the at least one second blood sugar level), which is measured using the first sensor when a specified time elapses after the specified event eb′ is detected, is less than or equal to a threshold, the processor may change the measurement cycle Tb of the first sensor from the second cycle ΔT2 to the first cycle ΔT1. For example, if the user's blood sugar level, which is measured using the first sensor at a time point (e.g., the 9th time point td9) after a specified time (e.g., 2 hours) elapses after eating food, is restored to 140 mg/dl or less, the processor may change the measurement cycle of the first sensor back to the first cycle ΔT1.

According to an embodiment of the disclosure, a wearable electronic device (e.g., the electronic device 200 in FIG. 2) may include a first sensor (e.g., the first sensor 210 in FIG. 2), a second sensor (e.g., the second sensor 220 in FIG. 2), a memory (e.g., the memory 250 in FIG. 2), and a processor (e.g., the processor 240 in FIG. 2) operatively connected to the first sensor, the second sensor, and the memory, wherein the processor may be configured to measure a blood sugar level of a user in a first cycle using the first sensor so as to store a first blood sugar level measured at a first time point according to the first cycle in the memory, detect a specified event, based on measured values received from the second sensor from a second time point after the first time point to a third time point, if the specified event is detected, measure a blood sugar level of the user in a second cycle different from the first cycle using the first sensor so as to store at least one second blood sugar level measured from a fourth time point to a fifth time point according to the second cycle in the memory, and calculate at least one feature value related to blood sugar of the user, based on the first blood sugar level and the at least one second blood sugar level.

The processor may be configured to configure an initial value of a change in the blood sugar of the user according to the specified event as the first blood sugar level.

The specified event may correspond to an event indicating that the user starts to eat food.

The second cycle may be shorter than the first cycle.

The processor may be configured to compare the at least one feature value with a threshold and, based on a result of comparing the at least one feature value with the threshold, change a measurement cycle of the first sensor to a third cycle different from the second cycle.

The third cycle may be shorter than the second cycle.

The processor may be configured to change a measurement cycle of the first sensor from the second cycle to the first cycle if a third blood sugar level of the user, which is measured using the first sensor when a specified time elapses after the specified event is detected, is less than or equal to a threshold.

The processor may be configured to compare the at least one feature value with a threshold and, based on a result of comparing the at least one feature value with the threshold, determine whether or not to provide a notification related to the blood sugar of the user.

The notification may include at least one piece of information about the user's health condition and information related to a diet or exercise guide determined based on the at least one feature value.

The at least one feature value may include at least one of a current blood sugar level, a fasting blood sugar level, a rate of change in blood sugar, a direction of change in blood sugar, the blood sugar response area, a slope of a blood sugar rise curve, a maximum blood sugar level, a change in blood sugar before and after eating food, the time required to return to the fasting blood sugar after eating food, the number of blood sugar peaks, or occurrence intervals of food intake events.

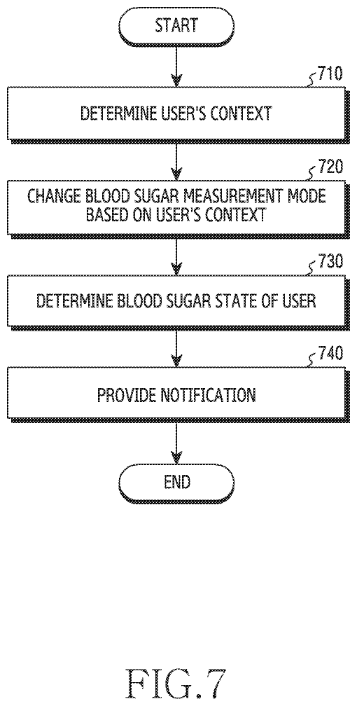

FIG. 7 is a flowchart illustrating a method of monitoring blood sugar, based on a user's context, according to an embodiment of the disclosure.

In the following embodiments, respective operations may be performed sequentially but are not necessarily limited thereto. For example, the sequence of the respective operations may vary, or at least two operations may be performed in parallel.

Referring to FIG. 7, operations 710 to 740 may be understood to be performed by a processor (e.g., the processor 240 in FIG. 2) of an electronic device (e.g., the electronic device 200 in FIG. 2).

Referring to FIG. 7, in operation 710, a processor (e.g., processor 240 in FIG. 2) of an electronic device (e.g., the electronic device 200 in FIG. 2) may determine a user's context. The processor may determine the user's context, based on a measured value of a second sensor (e.g., the second sensor 220 in FIG. 2). The user's context may include, for example, at least one of biometric information of the user, a user's situation, or a user's external environment. The user's context related to blood sugar monitoring may include at least one of a fasting state, a food intake state, and a pregnancy state.

In operation 720, the processor may change (or configure) a blood sugar measurement mode, based on the user's context. For example, if the user's context is determined to be a fasting state, the processor may configure (or change) the blood sugar measurement mode to a fasting blood sugar test mode. In the fasting blood sugar test mode, the processor may measure a fasting blood sugar level of the user using a first sensor (e.g., the first sensor 210 in FIG. 2). As another example, if the user's context is determined to be a food intake state, the processor may configure (or change) the blood sugar measurement mode to a glucose tolerance test mode. In the glucose tolerance test mode, the processor may measure a blood sugar level of the user during the food intake and a postprandial blood sugar level of the user using the first sensor. As another example, if the user's context is determined to be a pregnancy state, the processor may configure (or change) the blood sugar measurement mode to a gestational diabetes test mode (or arbitrary test mode). In the gestational diabetes test mode (or arbitrary test mode), the processor may measure a blood sugar level of the user who is pregnant using the first sensor.

In operation 730, the processor may determine the blood sugar state of the user. The processor may determine the blood sugar state of the user, based on the user's blood sugar level according to the user's context. For example, if the fasting blood sugar level is 100 mg/dl or less in the fasting blood sugar test mode, the processor may determine that the user's blood sugar is normal. In addition, if the fasting blood sugar level is 100 mg/dl to 125 mg/dl in the fasting blood sugar test mode, the processor may determine that the user's blood sugar corresponds to prediabetes (or fasting blood sugar disorder). In addition, if the fasting blood sugar level is 126 mg/dl or more in the fasting blood sugar test mode, the processor may determine that the user's blood sugar corresponds to a diabetic state. As another example, if the postprandial blood sugar level is 140 mg/dl or less in the glucose tolerance test mode, the processor may determine that the user's blood sugar is normal. In addition, if the postprandial blood sugar level is 140 mg/dl to 199 mg/dl in the glucose tolerance test mode, the processor may determine that the user's blood sugar corresponds to a prediabetic (or glucose tolerance disorder) state. In addition, if the postprandial blood sugar level is 200 mg/dl or more in the glucose tolerance test mode, the processor may determine that the user's blood sugar corresponds to a diabetic state. As another example, if the fasting blood sugar level is 200 mg/dl or more in the gestational diabetes test mode (or arbitrary test mode), the processor may determine that the user's blood sugar corresponds to a diabetic state.

In operation 740, the processor may provide a notification. The processor may display at least one of the user's blood sugar level, information about the user's blood sugar state, and information related to a diet or exercise guide on a display (e.g., the display 230 in FIG. 2). According to an embodiment, the processor may transmit at least one of the user's blood sugar level, information about the user's blood sugar state, and information related to a diet or exercise guide to an external electronic device through a communication circuit (e.g., the communication circuit 260 in FIG. 2).

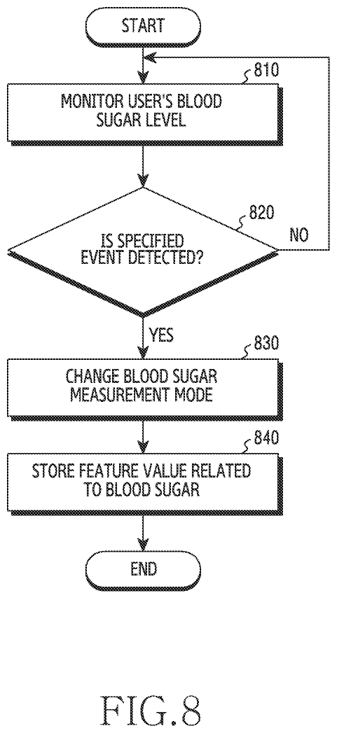

FIG. 8 is a flowchart illustrating a method of monitoring blood sugar when a specified event is detected according to an embodiment of the disclosure.

In the following embodiments, respective operations may be performed sequentially but are not necessarily limited thereto. For example, the sequence of the respective operations may vary, or at least two operations may be performed in parallel.

Referring to FIG. 8, operations 810 to 840 may be understood to be performed by a processor (e.g., the processor 240 in FIG. 2) of an electronic device (e.g., the electronic device 200 in FIG. 2).

Referring to FIG. 8, in operation 810, a processor (e.g., the processor 240 in FIG. 2) of an electronic device (e.g., the electronic device 200 in FIG. 2) may monitor a user's blood sugar level. For example, the processor may measure the user's blood sugar level in a specified cycle using a first sensor (e.g., the first sensor 210 in FIG. 2).

In operation 820, the processor may determine whether or not a specified event is detected. The processor may detect a specified event, based on measured values received from a second sensor (e.g., the second sensor 220 in FIG. 2). The specified event may correspond to an event indicating that the user starts to eat food (hereinafter referred to as a food intake start event).

If the specified event is not detected (NO in operation 820), the processor may return to operation 810 to monitor the user's blood sugar level.

If the specified event is detected (YES in operation 820), the processor may change (or configure) a blood sugar measurement mode in operation 830. For example, a blood sugar measurement mode for measuring the user's blood sugar level to monitor the user's blood sugar level before the specified event is detected may correspond to a fasting blood sugar test mode. In the fasting blood sugar test mode, the measurement cycle of the first sensor may be configure as a first cycle. If the specified event is detected, the blood sugar measurement mode may be changed from the fasting blood sugar test mode to a glucose tolerance test mode. In the glucose tolerance test mode, the measurement cycle of the first sensor may be changed from the first cycle to a second cycle. The second cycle may be shorter than the first cycle.

In operation 840, the processor may store a feature value related to blood sugar. The feature value related to blood sugar may include at least one of a current blood sugar level, a fasting blood sugar level, a rate of change in blood sugar, a direction of change in blood sugar, the blood sugar response area, a slope of a blood sugar rise curve, a maximum blood sugar level (or blood sugar peak value), a change in blood sugar before and after eating food, the time required to return to the fasting blood sugar after eating food, the number of blood sugar peaks, or occurrence intervals of food intake events.

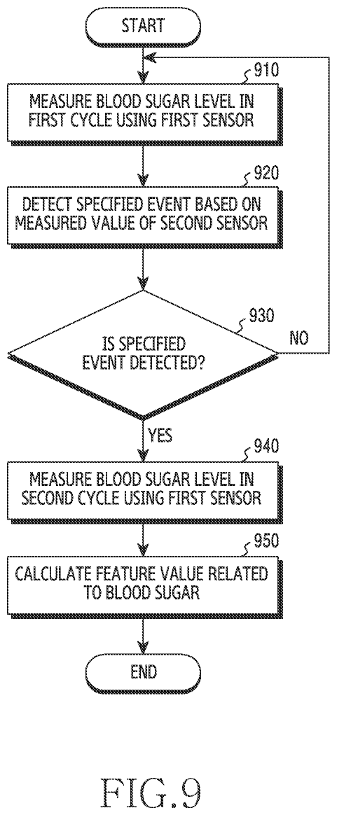

FIG. 9 is a flowchart illustrating a method of monitoring blood sugar when a specified event is detected according to an embodiment of the disclosure.

In the following embodiments, respective operations may be performed sequentially but are not necessarily limited thereto. For example, the sequence of the respective operations may vary, or at least two operations may be performed in parallel.

Referring to FIG. 9, operations 910 to 950 may be understood to be performed by a processor (e.g., the processor 240 in FIG. 2) of an electronic device (e.g., the electronic device 200 in FIG. 2).

Referring to FIG. 9, in operation 910, a processor (e.g., the processor 240 in FIG. 2) of an electronic device (e.g., the electronic device 200 in FIG. 2) may measure a user's blood sugar level in a first cycle using a first sensor (e.g., the first sensor 210 in FIG. 2). In addition, the processor may store a first blood sugar level measured at a first time point according to the first cycle in a memory (e.g., the memory 250 in FIG. 2). The first time point may be, for example, a time point in a user's fasting state. The first cycle may be, for example, a 15-minute cycle.

In operation 920, the processor may detect a specified event, based on a measured value of a second sensor (e.g., the second sensor 220 in FIG. 2). The processor may detect the specified event, based on measured values received from the second sensor from a second time point after the first time point to a third time point. The specified event may correspond to an event indicating that the user starts to eat food (hereinafter referred to as a food intake start event). The processor may detect an event subsequent to the specified event, based on the measured values received from the second sensor. The subsequent event may correspond to an event indicating that the user has finished eating food (hereinafter referred to as a food intake end event).

In operation 930, the processor may determine whether or not the specified event is detected. The processor may use a machine learning technique in order to detect an event related to food intake (e.g., the food intake start event and/or the food intake end event). For example, the processor may select structural feature points of a waveform of a measurable motion sensor from detailed actions in which an event related to food intake may occur and classify the food intake actions of the user using relative positions and states between the respective feature points. In this case, the processor may utilize data such as acceleration signal data measured during the user's action of eating food, change data in the direction of gravity generated while rotating the wrist, or a change in feature points of a gyro signal. In addition, the processor may use at least one of the average value, standard deviation, maximum value, minimum value, mode value, or median value for each axis of the acceleration sensor and/or the gyro sensor at minimum unit time (e.g., 1 minute) intervals in order to use statistical features. In addition, the processor may utilize various machine learning techniques such as TCN, DT, and KNN.

The processor may use a microphone sensor (e.g., the input module 150 in FIG. 1) in order to detect an event related to food intake (e.g., the food intake start event and/or the food intake end event). For example, the processor may obtain sound data generated when the user eats food using the microphone sensor and detect an event related to food intake using the sound data. As another example, the processor may detect an event related to food intake using sound data, which is generated when the user eats food, obtained using the microphone sensor and data on the movement of the user's temporomandibular joint while chewing the food measured by an acceleration sensor.

If it is determined that the specified event is not detected (NO in operation 930), the processor may return to operation 910 to reperform blood sugar measurement (operation 910) and detection of the specified event (operation 920).

If it is determined that the specified event is detected (YES in operation 930), the processor may measure a user's blood sugar level in a second cycle, which is different from the first cycle, using the first sensor in operation 940. For example, the processor may change (or adjust) a measurement cycle (e.g., a blood sugar measurement cycle) of the first sensor from the first cycle to the second cycle. In addition, the processor may store at least one second blood sugar level measured from a fourth time point to a fifth time point according to the second cycle in the memory (e.g., the memory 250 in FIG. 2). The period from the fourth time point to the fifth time point may be, for example, one of periods corresponding to the state in which the user is eating food and the state in which a predetermined time elapses after the user eats food. The second cycle may be shorter than the first cycle. The second cycle may be, for example, a 5-minute cycle.

In operation 950, the processor may calculate a feature value related to blood sugar. The processor may calculate at least one feature value related to the user's blood sugar, based on the first blood sugar level and the at least one second blood sugar level. The first blood sugar level is a blood sugar level measured in the first cycle at the first time point and may correspond to, for example, a fasting blood sugar level measured on an empty stomach. The at least one second blood sugar level is a blood sugar level measured in the second cycle from the fourth time point to the fifth time point and may correspond to, for example, a blood sugar level measured during food intake or a postprandial blood sugar level. The fourth time point at which the at least one second blood sugar level starts to be measured may be the same as the third time point or later than that in consideration of the time taken to detect the specified event. The specified event may be detected at a time point somewhat later than the time point at which the user actually starts to eat food. For example, the blood sugar level measured at the fourth time point may be the blood sugar level measured after the user has consumed food to some extent, instead of the blood sugar level at the time point when the user actually starts to eat food. Accordingly, in order to more accurately monitor a change in blood sugar according to the occurrence (or detection) of the specified event, the processor may configure an initial value of the change in blood sugar of the user according to the specified event as the first blood sugar level.

The at least one feature value related to the user's blood sugar may include at least one of a current blood sugar level, a fasting blood sugar level, a rate of change in blood sugar, a direction of change in blood sugar, the blood sugar response area, a slope of a blood sugar rise curve, a maximum blood sugar level (or blood sugar peak value), a change in blood sugar before and after eating food, the time required to return to the fasting blood sugar after eating food, the number of blood sugar peaks, or occurrence intervals of food intake events.

The processor may configure a threshold for the at least one feature value. In addition, the processor may compare the at least one feature value with the threshold and, based on a result of comparing the at least one feature value with the threshold, change the measurement cycle of the first sensor to a third cycle, which is different from the second cycle. If the at least one feature value exceeds the threshold, the processor may change the measurement cycle of the first sensor to a third cycle different from the second cycle. If the number of times the at least one feature value exceeds the threshold exceeds a specified number of times during a specified period, the processor may change the measurement cycle of the first sensor to a third cycle different from the second cycle. The third cycle may be shorter than the second cycle. The third cycle may be, for example, a 1-minute cycle.

If a third blood sugar level of the user measured using the first sensor is equal to or less than a threshold when a specified time elapses after the specified event is detected, the processor may change the measurement cycle of the first sensor from the second cycle to the first cycle. For example, if a third blood sugar level of the user measured using the first sensor is recovered to 140 mg/dl or less when a specified time (e.g., 2 hours) elapses after eating, the processor may change the measurement cycle of the first sensor back to the first cycle.

The processor may compare the at least one feature value with the threshold and, based on a result of comparing the at least one feature value with the threshold, determine whether or not to provide a notification related to the user's blood sugar. If the at least one feature value exceeds the threshold, the processor may determine whether or not to provide a notification related to the user's blood sugar. If the number of times the at least one feature value exceeds the threshold exceeds a specified number of times during a specified period, the processor may determine whether or not to provide a notification related to the user's blood sugar. The notification may include, for example, at least one piece of information about the user's health condition and information related to a diet or exercise guide determined based on the at least one feature value.

According to various embodiments of the disclosure, a blood sugar monitoring method may include measuring a blood sugar level of a user in a first cycle using a first sensor (e.g., the first sensor 210 in FIG. 2) so as to store a first blood sugar level measured at a first time point according to the first cycle in a memory (e.g., the memory 250 in FIG. 2) (e.g., the operation 910 in FIG. 9), detecting a specified event, based on measured values received from a second sensor (e.g., the second sensor 220 in FIG. 2) from a second time point after the first time point to a third time point (e.g., the operation 920 in FIG. 9), if the specified event is detected, measuring a blood sugar level of the user in a second cycle different from the first cycle using the first sensor so as to store at least one second blood sugar level measured from a fourth time point to a fifth time point according to the second cycle in the memory (e.g., the operation 940 in FIG. 9), and calculating at least one feature value related to blood sugar of the user, based on the first blood sugar level and the at least one second blood sugar level (e.g., the operation 950 in FIG. 9).

The calculating of the at least one feature value may include configuring an initial value of a change in the blood sugar of the user according to the specified event as the first blood sugar level.

The specified event may correspond to an event indicating that the user starts to eat food.

The second cycle may be shorter than the first cycle.

The blood sugar monitoring method may further include comparing the at least one feature value with a threshold and, based on a result of comparing the at least one feature value with the threshold, changing a measurement cycle of the first sensor to a third cycle different from the second cycle.

The third cycle may be shorter than the second cycle.

The blood sugar monitoring method may further include changing a measurement cycle of the first sensor from the second cycle to the first cycle if a third blood sugar level of the user, which is measured using the first sensor when a specified time elapses after the specified event is detected, is less than or equal to a threshold.

The blood sugar monitoring method may further include comparing the at least one feature value with a threshold and, based on a result of comparing the at least one feature value with the threshold, determining whether or not to provide a notification related to the blood sugar of the user.

The notification may include at least one piece of information about the user's health condition and information related to a diet or exercise guide determined based on the at least one feature value.

The at least one feature value may include at least one of a current blood sugar level, a fasting blood sugar level, a rate of change in blood sugar, a direction of change in blood sugar, the blood sugar response area, a slope of a blood sugar rise curve, a maximum blood sugar level, a change in blood sugar before and after eating food, the time required to return to the fasting blood sugar after eating food, the number of blood sugar peaks, or occurrence intervals of food intake events.

The electronic device according to various embodiments of the disclosure may be one of various types of electronic devices. The electronic devices may include, for example, a portable communication device (e.g., a smartphone), a computer device, a portable multimedia device, a portable medical device, a camera, a wearable device, or a home appliance. The electronic device is not limited to those described above.

It should be appreciated that various embodiments of the disclosure and the terms used therein are not intended to limit the technological features set forth herein to particular embodiments and include various changes, equivalents, or replacements for a corresponding embodiment. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

A method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

Each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable electronic device comprising:
a first sensor;
a second sensor;
a memory; and
a processor operatively connected to the first sensor, the second sensor, and the memory,
wherein the processor is configured to:
measure a blood sugar level of a user in a first cycle using the first sensor so as to store a first blood sugar level measured at a first time point according to the first cycle in the memory,
detect a specified event, based on measured values received from the second sensor from a second time point after the first time point to a third time point,
in case that the specified event is detected, measure the blood sugar level of the user in a second cycle different from the first cycle using the first sensor so as to store at least one second blood sugar level measured from a fourth time point to a fifth time point according to the second cycle in the memory, and
calculate at least one feature value related to blood sugar of the user, based on the first blood sugar level and the at least one second blood sugar level.

2. The wearable electronic device of claim 1, wherein the processor is further configured to configure an initial value of a change in the blood sugar of the user according to the specified event as the first blood sugar level.

3. The wearable electronic device of claim 1, wherein the specified event corresponds to an event indicating that the user starts to eat food.

4. The wearable electronic device of claim 1, wherein the second cycle is shorter than the first cycle.

5. The wearable electronic device of claim 1, wherein the processor is further configured to:
compare the at least one feature value with a threshold; and
based on a result of comparing the at least one feature value with the threshold, change a measurement cycle of the first sensor to a third cycle different from the second cycle.

6. The wearable electronic device of claim 5, wherein the third cycle is shorter than the second cycle.

7. The wearable electronic device of claim 1, wherein the processor is further configured to change a measurement cycle of the first sensor from the second cycle to the first cycle in case that a third blood sugar level of the user, which is measured using the first sensor when a specified time elapses after the specified event is detected, is less than or equal to a threshold.

8. The wearable electronic device of claim 1, wherein the at least one feature value comprises at least one of a current blood sugar level, a fasting blood sugar level, a rate of change in blood sugar, a direction of change in blood sugar, a blood sugar response area, a slope of a blood sugar rise curve, a maximum blood sugar level, a change in blood sugar before and after eating food, the time required to return to the fasting blood sugar after eating food, a number of blood sugar peaks, or occurrence intervals of food intake events.

9. A blood sugar monitoring method comprising:
measuring a blood sugar level of a user in a first cycle using a first sensor so as to store a first blood sugar level measured at a first time point according to the first cycle in a memory;
detecting a specified event, based on measured values received from a second sensor from a second time point after the first time point to a third time point;
in case that the specified event is detected, measuring the blood sugar level of the user in a second cycle different from the first cycle using the first sensor so as to store at least one second blood sugar level measured from a fourth time point to a fifth time point according to the second cycle in the memory; and
calculating at least one feature value related to blood sugar of the user, based on the first blood sugar level and the at least one second blood sugar level.

10. The blood sugar monitoring method of claim 9, wherein the calculating of the at least one feature value comprises configuring an initial value of a change in the blood sugar of the user according to the specified event as the first blood sugar level.

11. The blood sugar monitoring method of claim 9, wherein the specified event corresponds to an event indicating that the user starts to eat food.

12. The blood sugar monitoring method of claim 9, wherein the second cycle is shorter than the first cycle.

13. The blood sugar monitoring method of claim 9, further comprising:
comparing the at least one feature value with a threshold; and
based on a result of comparing the at least one feature value with the threshold, changing a measurement cycle of the first sensor to a third cycle different from the second cycle.

14. The blood sugar monitoring method of claim 13, wherein the third cycle is shorter than the second cycle.

15. The blood sugar monitoring method of claim 9, further comprising changing a measurement cycle of the first sensor from the second cycle to the first cycle in case that a third blood sugar level of the user, which is measured using the first sensor when a specified time elapses after the specified event is detected, is less than or equal to a threshold.

16. The wearable electronic device of claim 1, wherein the processor is further configured to:
compare the at least one feature value with a threshold; and
based on a result of comparing the at least one feature value with the threshold, determine whether or not to provide a notification related to the blood sugar of the user.

17. The wearable electronic device of claim 16, wherein the notification comprises at least one piece of information about the user's health condition and information related to a diet or exercise guide determined based on the at least one feature value.

18. The blood sugar monitoring method of claim 9, further comprising:
comparing the at least one feature value with a threshold; and
based on a result of comparing the at least one feature value with the threshold, determining whether or not to provide a notification related to the blood sugar of the user.

19. The blood sugar monitoring method of claim 18, wherein the notification comprises at least one piece of information about the user's health condition and information related to a diet or exercise guide determined based on the at least one feature value.

20. The blood sugar monitoring method of claim 9, wherein the at least one feature value comprises at least one of a current blood sugar level, a fasting blood sugar level, a rate of change in blood sugar, a direction of change in blood sugar, a blood sugar response area, a slope of a blood sugar rise curve, a maximum blood sugar level, a change in blood sugar before and after eating food, the time required to return to the fasting blood sugar after eating food, a number of blood sugar peaks, or occurrence intervals of food intake events.
